(19) United States
(12) Patent Application Publication
(10) Pub. No.: US 2009/0017174 A1
(43) Pub. Date: Jan. 15, 2009

(54) Food product treatment using alkaline electrolyzed water

(75) Inventor: Yen-Con Hung, Peachtree City, GA (US)

Correspondence Address: Mr. Edward J. Timmer, P.O.Box 770, Richland,, MI, 49083-0770, US

(73) Assignee: University of Georgia Research Foundation, Inc.

(21) Appl. No.: 12/220,315

(22) Filed: Jul. 23, 2008

Related U.S. Application Data
(63) Continuation-in-part of application No. 10/927,924, filed on Aug. 27, 2004.
(60) Provisional application No. 60/962,091, filed on Jul. 26, 2007.

(51) Int. Cl. A23L 3/32; A23L 1/48

(52) U.S. Cl. 426/237; 426/589; 426/654

(57) ABSTRACT

Treatment of food and agricultural products involves contacting the product by washing or otherwise with alkaline electrolyzed oxidizing water. Treatments include using brine solutions or marinades that include the alkaline electrolyzed oxidizing water therein.

### RELATED APPLICATIONS

This application is a continuation-in-part of Ser. No. 10/927,924 filed Aug. 27, 2004, and also claims priority and benefits of provisional application Ser. No. 60/962,091 filed Jul. 26, 2007.

### FIELD OF THE INVENTION

The present invention relates to a method for treating food and agricultural products using alkaline electrolyzed oxidizing water alone or as part of brine solutions or marinades.

### BACKGROUND OF THE INVENTION

Chemical deterioration like oxidative spoilage is of great interest to the food industry. For example, lipid oxidation in meat before cooking follows the lipid autoxidation scheme and affects its flavor and color of meat products. Antioxidants are considered to function as scavengers against reactive oxygen species or free radicals. A free radical is any molecule which contains one or more unpaired electrons. The most familiar antioxidants are vitamins A, C, E and some minerals. Many food compounds such as phytochemicals in plants and zoochemcials from animals are also believed to have strong antioxidant effects.

A process involving electrolysis of water can produce water with bioactive components. Reduced water produced by electrolysis (alkaline electrolyzed oxidizing water referred to as alkaline EO water) has a strong reduction potential, low dissolved oxygen and high dissolved hydrogen and functions as antioxidants due to neutralize reactive oxygen species. Oxidant water produced by electrolysis (acidic electrolyzed water referred to as acidic EO water) has a strong microbicidal effect.

Oxidation reduction potential (ORP) is an index to show the oxidizing effect and reducing effect of a liquid. If ORP is negative, the liquid has a reducing effect. Immediately after electrolysis, ORP of alkaline EO water is −800 mV or less. An electrolyzed alkaline aqueous solution has been considered to function as a scavenger against reactive oxygen species (J. Hanaoka et al. in Appl. Electro. 31: 1307-1313, 2001 and S. Shirahata et al. in Biochem. Biophysic. Res. Corn. 234: 269-274, 1997). Alkaline EO water also has been reported to be supersaturated with hydrogen and has a great redox potential (K. Kikuchi et al. in J. Appl. Electro. 31: 1301-1306, 2001). Several scientists have reported that hydrogen gas in the alkaline EO water exhibited antioxdative activities (K. Miyashita et al. in Biosci. Biotechnol. Biochem. 63:421-423, 1999 and S. Shirahata et al. in Biochem. Biophysic. Res. Corn. 234: 269-274, 1997)). In another study, it was reported that alkaline EO water completely inhibited the aqueous oxidation of polyunsaturated lipids such as ethyl linoleate and ethyl docosahexaenoate (K. Miyashita et al. in Biosci. Biotechnol. Biochem. 63: 421-423, 1999).

### SUMMARY OF THE INVENTION

The present invention provides a method for treatment of food and agricultural products using alkaline electrolyzed oxidizing (EO) water. The food and agricultural product can be contacted with the alkaline EO water for a time to achieve certain benefits described herein. The food and agricultural products include, but are not limited to, meat, poultry, hot dogs, produce such as vegetables and fruit, and nuts, seafood, and cut flowers.

In an illustrative embodiment of the invention, alkaline EO water is used to remove heme and non-heme pigments and fat of mechanically separated chicken meat (MSC) during contacting of the MSC and the alkaline EO water. The strong reduction potential and oxygen radical absorbance capacity of alkaline EO water can prevent lipid oxidation of the washed chicken meat during transportation and storage. EO water also prevents lipid oxidation of brine solutions for hot dogs chilling operation.

The present invention also provides a method for making marinade wherein the marinade includes alkaline electrolyzed oxidizing (EO) water as an ingredient of the marinade.

The present invention still further provides a brine solution that includes alkaline electrolyzed oxidizing (EO) water. The brine solution including the alkaline electrolyzed oxidizing (EO) water can be employed to chill hot dogs after cooking.

Advantages of the present invention will become more readily apparent from the following description taken with the following drawings.

### BRIEF DESCRIPTION OF THE DRAWINGS

FIG. 1 is schematic longitudinal sectional view of a slab type, membrane electrolytic cell for use in practice of the invention.

FIG. 2 is a cross-sectional view of FIG. 1 taken along lines 2-2.

FIGS. 3 and 4 are schematic views of other electrolytic cells for use in practice of the invention.

FIG. 5 is an exploded view of a particular emplary electrolytic cell of the type shown in FIG. 1 for use in practice of the invention. FIG. 5A is a perspective view of the opposite side of the outer end frame.

### DETAILED DESCRIPTION OF THE INVENTION

The present invention provides in one embodiment a method for treatment of food and agricultural products using alkaline electrolyzed oxidizing (EO) water. The food and agricultural product can be contacted with the alkaline EO water for a time to achieve certain benefits and include, but are not limited to, meat, poultry, fresh produce such as vegetables and fruit, and nuts, seafood, and cut flowers.

The alkaline EO water used in practice of the invention can be produced using any available design of the electrolysis process with any electrolytic cell as long as the alkaline EO water has the properties described in the next paragraph. An electrolytic cell and process useful in practicing the invention are described below under Electrolytic Cell and are illustrated in the figures described below.

In practice of the invention, the alkaline EO water can have a pH in the range of about 8 to about 12, an ORP in the range of −100 mV to −1000 mV, and temperature in the range of 32 to 80 degrees F. The alkaline EO water can be produced at the cathode(s) of the electrolytic cell, or at the anode(s) of the electrolytic cell if the pH can be modified to be in the range of about 8 to about 12.

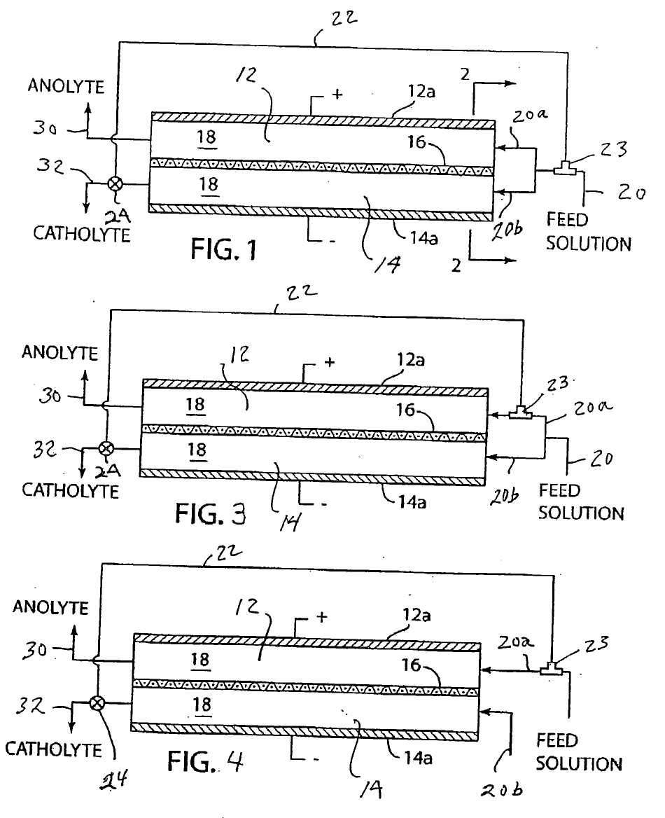

An exemplary alkaline EO water had the following properties after electrolysis: pH=11 and ORP of −800 mV in the temperature range. This exemplary akaline EO water was produced using an electrolytic cell of the type shown in FIG. 1 and operated using the following parameters: The feed water comprised an aqueous 0.1 weight % NaCl solution. The feed water was electrolyzed at a cell voltage of 10V and at a temperature of 24 degrees C. plus or minus 1 degree C. The feed water was supplied to the cathode and anode chambers 14 collectively at a flow rate of 1.0 L/minute with no recycling of catholyte to the anode chambers. Alkaline catholyte (pH above 11) from the cathode chambers 14 (FIG. 1) was collected for the chicken meat treatment tests described next.

Mechanically separated chicken meat (MSC) was treated either by spraying the carcasses before mechanical meat separation or by mixing the MSC with the exemplary alkaline EO water at room or refrigerated temperature for 30 seconds to 5 minutes with or without blending. Blending was effected by placing the MSC and the alkaline EO water in a bag and shaking the bag. Excess alkaline EO water was removed from the MSC by centrifugal force.

The treatment of the chicken meat was found to remove heme and non-heme pigments and fat of the mechanically separated chicken meat (MSC) during washing. The strong reduction potential and oxygen radical absorbance capacity of alkaline EO water can prevent lipid oxidation of the washed chicken meat during transportation and storage. Alkaline EO water washed MSC has a lighter color and longer shelf life than un-washed MSC. Thus, an illustrative commercial application of the invention involves treating MSC with alkaline EO water.

Another commercial application of the invention involves using alkaline EO water in brine solution employed to chill hot dogs after cooking. The brine solution with alkaline EO water pursuant to the invention can remove fat and protein from the surface of the hot dogs. Alkaline EO water can also enhance the stability of the brine solution by preventing the oxidation of lipids in the brine solution and enhance the quality of hot dogs by preventing the development of off-flavor of the brine solution.

Still another commercial application of the invention involves using the alkaline EO water to prepare marinade products to prevent oxidation reactions during marination. The alkaline EO water is incorporated as an ingredient in the preparation of the mainade itself. The present invention thereby envisions a method for making marinade as well as the marinade wherein the marinade includes alkaline electrolyzed oxidizing (EO) water as an ingredient of the marinade.

### Electrolytic Cell

The alkaline EO water used in practice of the invention can be produced using any available design of the electrolysis process with any electrolytic cell as long as the alkaline EO water has the properties described in the paragraphs above.

For purposes of illustration and not limitation, FIGS. 1-4 and FIGS. 5 and 5A are now discussed to illustrate electrolytic cells which can be used in practice of the invention. Commercially available electrolytic cells which can be used in practice of the invention to produce the alkaline EO water include, but are not limited to, ROX-10WB, ROX-20TB, ROX-20TX, HOX-40A manufactured by Hoshizaki Electric Inc.; EA-300 and EA-500 manufactured by Aquastel; α-light and α-2000N manufactured by Amano; AQ-2000 manufactured by Sterilox Technologies, Inc.; NDX-65KMH manufactured by Proton Labs.

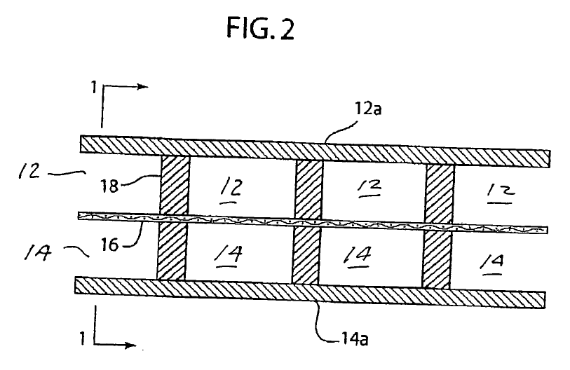

Referring to FIGS. 1-2, apparatus for electrolyzing water in practice of the invention is schematically shown. The apparatus includes a plurality of anode chambers 12 and cathode chambers 14 separated by a membrane 16. The chambers 12, 14 have a rectangular cross-section when shown in cross-sectional FIG. 2, although the chambers can have any shape. One or more flat, plate-like anode electrodes 12a and cathode electrodes 14a (one of each electrode shown) are disposed in the anode chambers and cathode chambers, respectively. The anode and cathode electrodes can comprise titanium or titanium coated with a precious metal, such as platinum, or they can comprise any other suitable electrode material. The membrane 16 can comprise either a non-ion selective separator membrane comprising, for example, nonwoven polyester fabric, or an ion-selective permeable membrane comprising, for example, a perfluorosulfonate ionomer. When the feed water to be electrolyzed comprises a dilute aqueous NaCl salt solution, the membrane allows Na+ ions to move toward the cathode electrode 14a from the anode chamber 12 and Cl⁻¹ ions to move toward the anode electrode 12a from the cathode chamber 14. The membrane 16 is spaced between the electrodes by electrically insulating plastic spacers 18. The electrodes are connected to a conventional electrical power supply (not shown) to thereby provide an electrochemical cell for electrolyzing to water.

The feed water to be electrolyzed can comprise a dilute aqueous NaCl solution, such as 0.01% to 25% by weight NaCl solution, although the invention can be practiced to electrolyze other aqueous solutions of KCl, MgCl₂ and other salts.

Referring to the embodiment of FIGS. 1-2, the feed water (designated “feed solution” in FIGS. 1, 3, and 4) is supplied to both the anode chambers 12 and cathode chambers 14 via a feed water supply conduit 20 that is branched to have an anode supply conduit section 20a and cathode supply section 20b from the common conduit 20. The anode supply water conduit section 20a supplies the feed water only to anode chambers 12 via a manifold (not shown) that communicates with each of the plurality of anode chambers 12 as described below for the example. The cathode supply water conduit section 20b supplies the feed water only to cathode chambers 14 via a manifold (not shown) that communicates with each of the plurality of cathode chambers 14 as described below for the example.

The feed water is cathodically electrolyzed in the cathode chambers 14 to produce EO water as alkaline catholyte. The feed water is anodically electrolyzed in the anode chambers 12 to produce electrolyzed (EO) water as anolyte whose pH can be modified for use in the invention. The pH-modified, anodically electrolyzed water (anolyte) is discharged from the anode chambers 12 by way of an anolyte discharge conduit 30 for collection and use. The cathodically electrolyzed water (catholyte) is discharged from the cathode chambers 14 by way of a catholyte discharge conduit 32 for collection and use and for recycling back to the anode chambers 12. In practice of the embodiment of the invention for the treating tests of the mechanically separated chicken meat (MSC) described above, almost all of the cathodically electrolyzed water was collected from conduit 32 for treatment the MSC (that is; substantially no catholyte was recycled back to the anode chambers).

However, the invention envisions using pH-modified anodically electrolyzed water (anolyte) if its pH can be modified to be in the range of about 8 to about 12. To this end, the apparatus includes catholyte return conduit 22 or other means for returning a portion of the alkaline catholyte from the cathode chamber 14 to the feed water in supply conduit 20 to provide a blend of the feed water and the catholyte to the anode chambers 12. The blend of feed water and catholyte is also provided to the cathode chambers 14. The flow rate of feed water in the cathode chambers 14 typically is substantially equal to the flow rate of the blend of feed water and recycled catholyte in the anode chambers 12, although different flow rates can be provided through the chambers 12, 14. A conventional valve 24 made of PVC is provided between the catholyte discharge conduit 32 and the catholyte return conduit 22 to control the flow rate of catholyte returned to the feed water. The return conduit 22 can be communicated to the supply conduit 20 by use of a conventional T-junction 23 or any other suitable pipe connecting means. The flow rate of the alkaline catholyte recycled or returned from the catholyte discharge conduit 32 to the feed water in supply conduit 20 is controlled to control pH of the anodically electrolyzed water (anolyte) in anode chamber 12 to provide a pH value of about 8 to about 12.

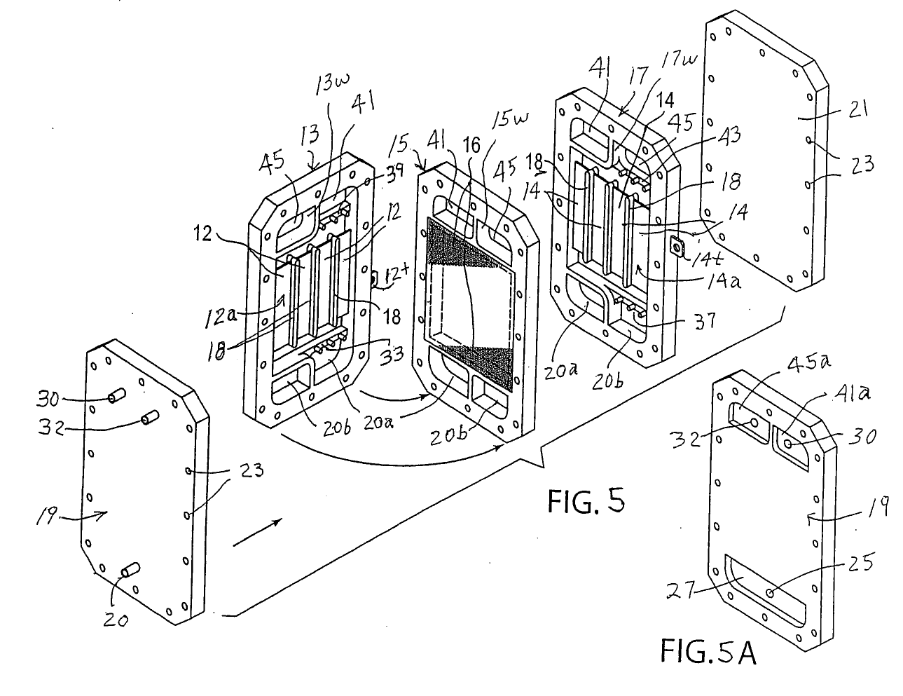

FIG. 3 illustrates another apparatus for use in practice of the invention wherein the catholyte return conduit 22 is communicated only to the supply branch 20a so as to supply the blend of feed water and recycled catholyte to the anode chambers 12 only. That is, the anode chambers 12 receive the blend of feed water and catholyte, while the cathode chambers 14 receive only feed water (sans recycled catholyte). FIG. 4 illustrates still another apparatus for use in practice of the invention wherein a separate feed water supply conduit 20a, 20b is provided to the anode chambers 12 and to the cathode chambers 14, and the catholyte return conduit 22 is communicated only to the supply conduit 20a to supply the blend only to the anode chambers 12.

Apparatus functioning as shown and described for FIG. 1 is shown in more detail in FIGS. 5, 5A. In FIGS. 5 and 5A, the side edges of a flat anode electrode 12a are mounted in a plastic frame 13 as shown, the membrane 16 is mounted (glued) on a plastic frame 15, and the side edges of a flat cathode electrode 14a are mounted in a plastic frame 17. The membrane 16 is shown broken away in FIG. 5 for convenience but spans the entire area between membrane segments shown. The spacers 18 are glued on opposite flat major surfaces of the anode electrode 12a and the cathode electrode 14a as shown. The frames 13, 15, 17 are adapted to be stacked side-by-side with end frames 19, 21 to form the electrolyzer. The frames includes holes 23 that are aligned when the frames are assembled to receive fasteners to hold and seal mating surfaces of the frames together. The assembled frames forming the electrolyzer were oriented vertically for experiments described below, but the orientation can be any other orientation than vertical.

When assembled, the end frame 19 includes feed water supply conduit 20 on its exterior side communicated in flow relation to a passage 25 therethrough to a manifold 27 on its interior side, FIG. 5A. The manifold 27 is communicated in flow relation to passages 20a, 20b that correspond to anode supply water conduit section 20a and cathode supply water conduit section 20b of FIG. 1 and that distribute feed water to the anode chambers 12 and to the cathode chambers 14, respectively. The feed water flows from the manifold 27 through passage 20a and channel 33 in frame 13 to the anode chambers 12, which are defined by flat anode electrode 12a, spacers 18, membrane 16 and sides of frame 13. The feed water flows through passage 20b and channel 37 in frame 17 to the cathode chambers 14, which are defined by flat cathode electrode 14a, spacers 18, membrane 16 and sides of frame 17, the cathode chambers 14 being on the opposite side of the membrane 16 from the anode chambers 12. The anolyte flows along and from the anode chambers 12 through channel 39 between the spacer posts shown to anolyte discharge passage 41, which is communicated in flow relation to anolyte discharge recess 41a on the interior of end frame 19 and then to anolyte discharge conduit 30. The catholyte flows along and from the cathode chambers 14 through channel 43 between the spacer posts shown to catholyte discharge passage 45, which is communicated in flow relation to catholyte discharge recess 45a on the interior of end frame 19 and then to catholyte discharge conduit 32. The anolyte discharge passage 41 is separated from the catholyte discharge passage 45 by walls 13w, 15w, 17w on frames 13, 15, 17, the walls being sealed to one another when the frames are assembled. The anode electrode 12a and cathode electrode 14a include respective integral positive and negative terminal tabs 12t and 14t embedded in and extending out of side of the frames where the electrical power is supplied. End frame 19, frames 13, 15, 17, and end frame 21 were stacked together to form the electrolyzer. The catholyte return conduit 22 (not shown in FIG. 5) was connected to catholyte discharge conduit 32 and to the feed water supply conduit 20 as shown in FIG. 1. Additional frames 13, 15, and 17 can be stacked together to increase production rate of electrolyzed water.

The invention can be practiced to wash or otherwise treat food and agricultural products, that include, but are not limited to, meat, poultry, fresh produce such as vegetables and fruit, and nuts, seafood, and cut flowers.

Although the invention has been described in terms of specific embodiments thereof, those skilled in the art will appreciate that the invention is not limited and changes and modifications can be made therein within the scope of the invention as set forth in the appended claims.

I claim:

1. A method of treating a food product or agricultural product, comprising contacting the product with alkaline electrolyzed oxidizing water for a time.

2. The method of claim 1 wherein the product is washed using the alkaline electrolyzed oxidizing water.

3. The method of claim 1 wherein the alkaline electrolyzed oxidizing water has a pH in the range of about 8 to about 12.

4. The method of claim 1 wherein the alkaline electrolyzed oxidizing water is produced at a cathode.

5. The method of claim 1 wherein the alkaline electrolyzed oxidizing water is pH-modified water produced at an anode.

6. The method of claim 3 wherein the alkaline electrolyzed oxidizing water has an ORP in the range of about −100 mV to about −1000 mV.

7. The method of claim 1 wherein the product is contacted with the alkaline electrolyzed oxidizing water at a temperature of about 32 to about 80 degrees F.

8. The method of claim 1 that extends the shelf life of the product

9. The method of claim 1 wherein the product includes meat, poultry, hot dogs, vegetables, fruit, nuts, seafood, or cut flowers.

10. A method of treating hot dogs, comprising contacting the hot dogs with alkaline electrolyzed oxidizing water for a time.

11. The method of claim 10 wherein the alkaline EO water is present in a brine solution employed to chill the hot dogs after cooking.

12. A method of making a marinade, comprising including alkaline electrolyzed oxidizing water as an ingredient in the marinade.

13. A brine solution comprising alkaline electrolyzed oxidizing water therein.

14. A marinade comprising alkaline electrolyzed oxidizing water therein.
